(19) United States
(12) Patent Application Publication
(10) Pub. No.: US 2021/0005294 A1
(43) Pub. Date: Jan. 7, 2021

(54) HEALTHCARE HUB

(71) Applicant: HealthTap, Inc., Mountain View, CA (US)

(72) Inventors: Sean K. Mehra, San Francisco, CA (US); William Gossman, Seattle, WA (US)

(21) Appl. No.: 16/597,157

(22) Filed: Oct. 9, 2019

Related U.S. Application Data
(60) Provisional application No. 62/869,171, filed on Jul. 1, 2019.

(51) Int. Cl. G16H 10/60; G16H 20/00; G16H 40/20

(52) U.S. Cl. CPC G16H 10/60; G16H 40/20; G16H 20/00

(57) ABSTRACT

A facility for providing healthcare assistance is described. The facility receives a symptom triage request from a first user. The facility provides to the first user a symptom triage response that is responsive to the received symptom request. The facility receives a prescription fulfillment request from a second user. The facility notifies notifying the second user of prescription fulfillment that is responsive to the received prescription fulfillment request.

### CROSS-REFERENCE TO RELATED APPLICATIONS

This application claims the benefit of provisional U.S. Patent Application No. 62/869,171, filed Jul. 1, 2019 and entitled “Healthcare Hub,” which is hereby incorporated by reference in its entirety.

This application is related to U.S. application Ser. No. 14/320,241, filed Jun. 30, 2014, now pending, which claims priority to U.S. Provisional Application No. 61/841,145, filed Jun. 28, 2013, U.S. Provisional Application No. 61/841,151, filed Jun. 28, 2013 and U.S. Provisional Application No. 61/841,174, filed Jun. 28, 2013, the contents of each of which are incorporated herein by reference.

This application is related to U.S. application Ser. No. 15/298,099, filed Oct. 19, 2016, which claims priority to U.S. Provisional Application No. 62/243,516, filed Oct. 19, 2015 and which is a continuation-in-part of U.S. application Ser. No. 14/320,241, filed Jun. 30, 2014, now pending, which claims priority to U.S. Provisional Application No. 61/841,145, filed Jun. 28, 2013, U.S. Provisional Application No. 61/841,151, filed Jun. 28, 2013 and U.S. Provisional Application No. 61/841,174, filed Jun. 28, 2013, the contents of each of which are incorporated herein by reference.

This application is related to U.S. application Ser. No. 15/839,595, filed Dec. 12, 2017 and is a continuation of Ser. No. 15/298,099, filed Oct. 19, 2016, which claims priority to U.S. Provisional Application No. 62/243,516, filed Oct. 19, 2015 and which is a continuation-in-part of U.S. application Ser. No. 14/320,241, filed Jun. 30, 2014, now pending, which claims priority to U.S. Provisional Application No. 61/841,145, filed Jun. 28, 2013, U.S. Provisional Application No. 61/841,151, filed Jun. 28, 2013 and U.S. Provisional Application No. 61/841,174, filed Jun. 28, 2013, the contents of each of which are incorporated herein by reference.

This application is related to U.S. application Ser. No. 15/851,189, filed Dec. 21, 2017, which is a divisional of U.S. application Ser. No. 14/320,241, filed Jun. 30, 2014, now pending, which claims priority to U.S. Provisional Application No. 61/841,145, filed Jun. 28, 2013, U.S. Provisional Application No. 61/841,151, filed Jun. 28, 2013 and U.S. Provisional Application No. 61/841,174, filed Jun. 28, 2013, the contents of each of which are incorporated herein by reference.

This application is related to U.S. application Ser. No. 15/851,211, filed Dec. 21, 2017, which is a divisional of U.S. application Ser. No. 14/320,241, filed Jun. 30, 2014, now pending, which claims priority to U.S. Provisional Application No. 61/841,145, filed Jun. 28, 2013, U.S. Provisional Application No. 61/841,151, filed Jun. 28, 2013 and U.S. Provisional Application No. 61/841,174, filed Jun. 28, 2013, the contents of each of which are incorporated herein by reference.

This application is related to U.S. application Ser. No. 15/851,226, Dec. 21, 2017, which is a divisional of U.S. application Ser. No. 14/320,241, filed Jun. 30, 2014, now pending, which claims priority to U.S. Provisional Application No. 61/841,145, filed Jun. 28, 2013, U.S. Provisional Application No. 61/841,151, filed Jun. 28, 2013 and U.S. Provisional Application No. 61/841,174, filed Jun. 28, 2013, the contents of each of which are incorporated herein by reference.

This application is related to U.S. Provisional Application No. 62/722,044, filed Aug. 13, 2018, the contents of which is incorporated herein by reference.

This application is related to U.S. application Ser. No. 16/239,495, filed Jan. 3, 2019, which is a continuation-in-part of Ser. No. 15/298,099, filed Oct. 19, 2016, which claims priority to U.S. Provisional Application No. 62/243,516, filed Oct. 19, 2015 and which is a continuation-in-part of U.S. application Ser. No. 14/320,241, filed Jun. 30, 2014, now pending, which claims priority to U.S. Provisional Application No. 61/841,145, filed Jun. 28, 2013, U.S. Provisional Application No. 61/841,151, filed Jun. 28, 2013 and U.S. Provisional Application No. 61/841,174, filed Jun. 28, 2013, the contents of each of which are incorporated herein by reference.

In cases where the present patent application conflicts with an application or other document incorporated herein by reference, the present application controls.

### BACKGROUND

Healthcare services of a variety of types can meaningfully improve the quality of patients' lives.

### BRIEF DESCRIPTION OF THE DRAWINGS

FIG. 1 is a network diagram showing an environment in which the facility operates in some embodiments.

FIG. 2 is a block diagram showing some of the components typically incorporated in at least some of the computer systems and other devices on which the facility operates.

FIG. 3 is a flow diagram showing a process performed by the facility in some embodiments to provide services to patients via a healthcare hub.

FIG. 4 is a feature diagram showing components included in the facility in various embodiments, and services provided by the facility in various embodiments.

FIG. 5 is an integration diagram showing details of how the facility permits the hub to be integrated with the efforts of partners and/or other third parties.

FIG. 6 is an ontology diagram showing a consumer health natural language ontology used by the facility in some embodiments.

### DETAILED DESCRIPTION

The inventors have recognized that many people have inadequate access to healthcare services and related resources. For example, the number of people who have access to third-party health insurance—1.1 billion—is only 31% of the 3.5 billion who have access to the Internet. Further, Pew Research Center found that fewer than 50% of those who access online health information find it helpful. Inadequate access to healthcare services and related resources is exacerbated by the lack of means for a user to interact with a variety of healthcare services in a unified and comprehensive manner.

In order to address these low levels of access to healthcare services and related resources, the inventors have conceived and reduced to practice a hardware and/or software facility for providing heterogeneous healthcare services via the Internet using an electronic hub (“the facility”). The facility receives and services a wide variety of requests from users, including, for example, patient users needing healthcare assistance, provider users providing healthcare assistance, and administrative users configuring the operation of the hub. In some embodiments, the facility integrates at least the following aspects of functionalities in a single application or executable: 1) access to informational answers to health questions or requests from users, 2) intelligent guidance to the right care based on user symptoms, 3) connecting with a health care service provider (e.g., virtually or in person), 4) receiving physician's orders, 5) encouragement to complete physician's plan, 6) ability to purchase and/or consume related healthcare goods and/or services (e.g., generic OTC drug, at home test, OTC treatment or device, etc.), and 7) ability to personalize some or all aspects of the health care service access to inform more responsive and more-cost effective user experience based on a unified, up-to-date medical record.

In many cases, servicing a received request involves routing the request. In various embodiments, the request can be routed to a native specialized component of the platform, or to an external resource, such as a partner's automated system, or a human provider employed by or otherwise affiliated with a partner. The hub receives a response to the routed request, and determines whether to return it to the requesting user, further route or process the response, or start over in processing the request.

In some embodiments, the facility operates in a mode where it engages in an interactive conversation with a user. In this mode, any single interaction or series of interactions can be performed by various hub components natively, or performed by external resources, switching among these mid-conversation without appearing to the user.

In some embodiments, the facility operates autonomously or semi-autonomously, taking action not directly triggered by any user request. This can involve, for example, following up with a user about a specialist referral or the administration of a dose of medicine; marketing a new capability to a user; requesting healthcare advice from a provider user; etc.

For example, patients can use the hub to pose and receive answers to healthcare questions, such as in an interactive real-time interaction with an intelligent chatbot; be instantly and intelligently guided to appropriate care; easily make payments in a way that is coordinated with their insurance workflows; and receive prescriptions, treatment plans, and reminders.

For providers, the hub aggregates demand for their services, helps them manage patient relationships, and serve patients at scale at a very low marginal cost.

The facility establishes a virtuous circle in which patients and providers find their successful interactions with the hub and the underlying services and resources rewarding, and are motivated to continue and extend their participation in the hub.

By performing in some or all of the foregoing ways, embodiments of the facility enable convenient access to valuable healthcare information to anyone with Internet access; efficiently match supply and demand; and/or provide seamless fulfillment of healthcare services by a variety of providers.

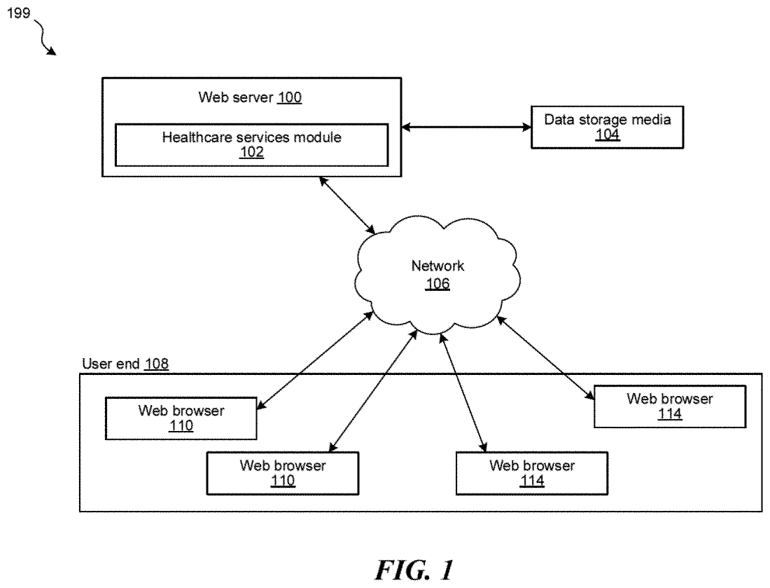

FIG. 1 is a network diagram showing an environment in which the facility operates in some embodiments. The environment 199 includes one or more user computer devices 110 (described herein as “patient computer devices”), one or more healthcare professional user computer devices 114 (sometimes described herein as “doctor computer devices”), a web server 100, and data storage media 104. A user end 108 (e.g., web browser displayable on a computer device) provides one or more user interfaces through which the users can interact with the hub provided by the facility. The web server 100 is electronically accessible through at least one network 106 to multiple computer devices (e.g., patient user computer devices 110, healthcare professional user devices 114). The network 106 may include any type of network infrastructure, such as the Internet, or any other wired, wireless and/or partially wired network.

The server 100 may be or include any database capable of storing and/or providing access to information, such as an electronic database, a computer and/or computerized server, database server or generally any network host capable of storing data and connected to any type of data network. Further, the server 100 may include or be a part of a distributed network or cloud computing environment. Any type of electronic and/or computerized device that is capable of storing information may be included as the server 100, and is considered within the scope of this disclosure. The server 100 may include computer-readable storage media, and a processor for processing data and executing algorithms, including any of the processes and algorithms set forth in this disclosure.

Web server 100 hosts, fully or partially, or otherwise accesses multiple application logic components (e.g., a Healthcare Services Module 102). As is understood by persons skilled in the relevant art, modules shown in FIG. 1 may represent sets of executable software instructions, as well as corresponding hardware (e.g., memory and processor) for executing the instructions. The server 100, Healthcare Services Module 102, and network 106 may include a variety of hardware and software components to provide successful functioning of the server 100 and the module 102, as is well-known within the art. Further, any features, characteristics, designs and/or functions that are known within the art may be included with the environment 199 to further enhance its efficiency.

The environment 199 may further include one or more patient computer devices 110 and one or more doctor computer devices 114. The patient computer devices 110 and doctor computer devices 114 may be any computerized devices capable of communicating with the server 100, for example via a network system 106. The one or more patient computer devices 110 may be operated by a patient user (which may be any user who votes using the environment 199 or otherwise seeks information related to professional users of the environment 199, and need not be an active patient of any treating physician or medical caregiver) of the environment 199, and the one or more doctor computer devices 114 may be operated by any doctor user of the environment 199 (which may be any user who may be evaluated using the environment 199, and may include any healthcare professional, such as a primary care physician, medical doctor, nurse, medical staff or other medical professional, or any representative thereof).

The data storage media 104 is a computer-readable storage medium, which may be any device or medium that can store code and/or data embodying or used by any one or more of the methods or functions described herein. This includes, but not limited to, volatile memory, non-volatile memory including by way of example semiconductor memory devices, e.g., Erasable Programmable Read-Only Memory (EPROM), Electrically Erasable Programmable Read-Only Memory (EEPROM), and flash memory devices, magnetic storage such as internal hard disks and removable disks and optical storage devices such as disk drives, magnetic tape, CDs (compact discs), DVDs (digital versatile discs or digital video discs), or other media capable of storing computer-readable media now known or later developed. The data storage media 104 may be included in, or electronically accessible by, the web server 100.

While the computer-readable medium is shown in some embodiments to be a single medium, the term “computer-readable medium” may include a single medium or multiple media (e.g., a centralized or distributed database, and/or associated caches and servers) that store the one or more instructions or data structures. The term “computer-readable medium” shall also be taken to include any tangible medium that is capable of storing, encoding or carrying instructions for execution by the computer and that cause the computer to perform any one or more of the methodologies of the present invention, or that is capable of storing, encoding or carrying data structures used by or associated with such instructions.

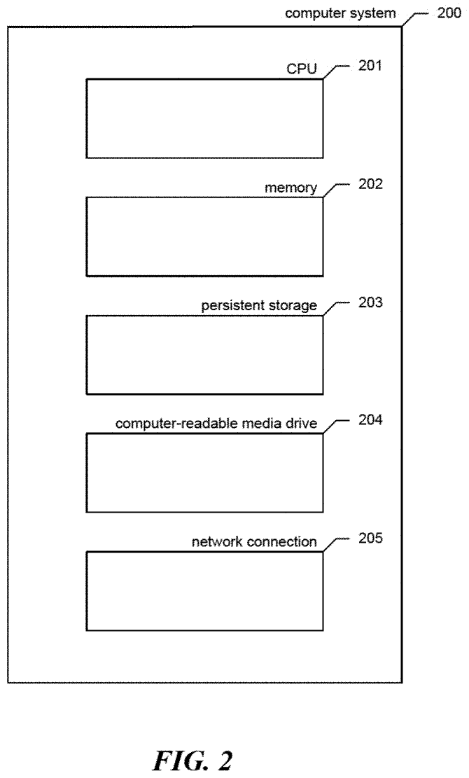

FIG. 2 is a block diagram showing some of the components typically incorporated in at least some of the computer systems and other devices on which the facility operates. In various embodiments, these computer systems and other devices 200 can include server computer systems, desktop computer systems, laptop computer systems, netbooks, mobile phones, personal digital assistants, televisions, cameras, automobile computers, electronic media players, etc. In various embodiments, the computer systems and devices include zero or more of each of the following: a central processing unit (“CPU”) 201 for executing computer programs; a computer memory 202 for storing programs and data while they are being used, including the facility and associated data, an operating system including a kernel, and device drivers; a persistent storage device 203, such as a hard drive or flash drive for persistently storing programs and data; a computer-readable media drive 204, such as a floppy, CD-ROM, DVD, or Blu-ray drive, for reading programs and data stored on a computer-readable medium; and a network connection 205 for connecting the computer system to other computer systems to send and/or receive data, such as via the Internet or another network and its networking hardware, such as switches, routers, repeaters, electrical cables and optical fibers, light emitters and receivers, radio transmitters and receivers, and the like. While computer systems configured as described above are typically used to support the operation of the facility, those skilled in the art will appreciate that the facility may be implemented using devices of various types and configurations, and having various components.

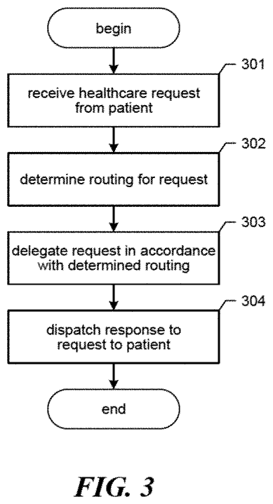

FIG. 3 is a flow diagram showing a process performed by the facility in some embodiments to provide services to patients via a healthcare hub. In act 301, the facility receives a healthcare request from or on behalf of a patient. In act 302, the facility determines a routing for the request. In various embodiments, the routing can involve routing the request to a particular function or code module; routing the request to a computer system operated by or on behalf of a partner; routing the request to a partner or other service provider, either programmatically, or via email, text message, voice call, facsimile, etc. In act 303, the facility delegates the request in accordance with the routing determined in act 302. In act 304, the facility dispatches a response to the request to the patient. In some embodiments, the facility dispatches the response using the same communication modality via which it received the request; in some embodiments, the facility uses a different modality to dispatch the response. After act 304, this process concludes.

Those skilled in the art will appreciate that the acts shown in FIG. 3 and in each of the flow diagrams discussed below may be altered in a variety of ways. For example, the order of the acts may be rearranged; some acts may be performed in parallel; shown acts may be omitted, or other acts may be included; a shown act may be divided into subacts, or multiple shown acts may be combined into a single act, etc.

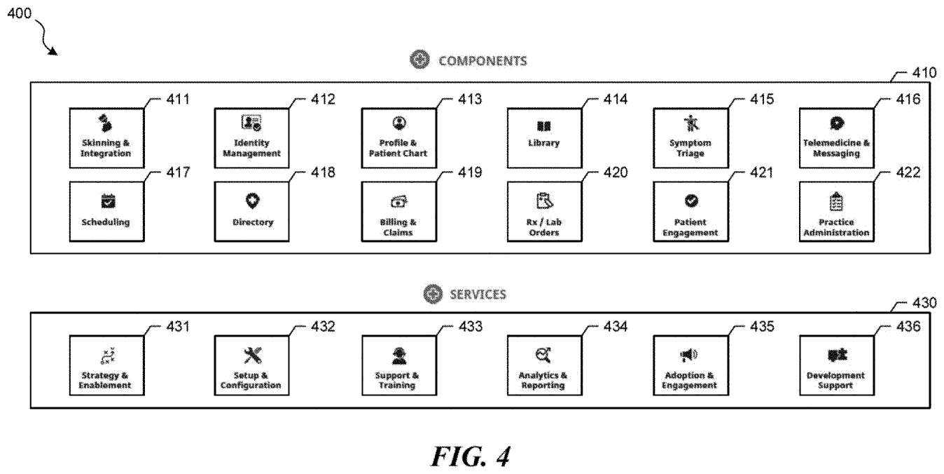

FIG. 4 is a feature diagram showing components included in the facility in various embodiments, and services provided by the facility in various embodiments. In various embodiments, the components 410 used by the facility include some or all of the following: a skinning and integration component 411; an identity management component 412; a profile and patient chart component 413; a library component 414; a system triage component 415; a telemedicine and messaging component 416; a scheduling component 417; a directory component 418; a billing and claims component 419; a prescription/laboratory orders component 420; a patient engagement component 421; and a practice administration component 422.

In some embodiments, the skinning and integration component 411 formats and presents information from different sources in a similar way in order to provide a cohesive user experience. In some embodiments, the skinning and integration component 411 can be used to adapt the appearance of the information provided via the facility for various purposes, such as to match the branding or trade dress of a customer who is labeling the hub with its own brand. In some embodiments, the identity management component 412 provides user authentication for interactions with the hub by users in various categories. In some embodiments, the profile and patient chart component 413 collects and manages information describing each patient, and each patient's electronic medical record. In some embodiments, the library component 414 provides various healthcare resources, such as articles; photos, charts, radiological studies, and other images; information about various healthcare institutions and providers; etc. In some embodiments, the symptom triage component 415 obtains information about a patient's symptoms—such as by interactive interviews—as a basis for predicting conditions or categories of conditions suffered by the patient, identifying appropriate providers and/or next steps toward treatment, etc. In some embodiments, the telemedicine and messaging component 416 provides mechanisms for interaction among users, in some cases between patient users and provider users. In some embodiments, the scheduling component 417 schedules events, such as appointments, tests, surgeries, check-ins, medicine dose administrations, etc. In some embodiments, the directory component 418 maintains and provides access to various directories, such as directories of providers, directories of institutions, etc. In some embodiments, the billing and claims component 419 manages aspects of billing patients and/or insurance companies for services, making payments in accordance with bills, etc. In some embodiments, the prescription/laboratory order component 420 manages interactions with pharmacies and labs, such as to present, fill, or refill prescriptions, or ordering lab tests, arranging details of their performance, and reporting the results. In some embodiments, the patient engagement component 421 takes various actions to promote use of the site to patients, such as responding one or more times to their inquiries; performing follow-ups; proactively advertising features of the hub that are likely relevant or useful to a particular patient based upon their profile or actions; etc. In some embodiments, the practice administration component 422 assists providers in organizing and operating their practices.

In various embodiments, the services 430 provided by the facility include one or more of a strategy and enablement service 431; a setup and configuration service 432; a support and training service 433; an analytics and reporting service 434; an adoption and engagement service 435; and/or a development support service 436. In some embodiments, the strategy and enablement service 431 allows operators of the hub to expand the hub to provide new services; existing services in different ways; different combinations of services; etc. In some embodiments, the setup and configuration service 432 assists operators of the hub in configuring it for use by a particular group of users, such as by providing information about the group; providing information about individual patients and/or providers; and/or tailoring various other aspects of the hub. In some embodiments, the support and training service 433 provides support in training about the hub for various categories of users, such as patients, providers, hub administrative users, etc. In some embodiments, the analytics and reporting service 434 analyzes patient and/or provider data and reports on the results. In some embodiments, the adoption and engagement service 435 helps market the hub to new and existing users of various types such as patients and providers, and monitors the result. In some embodiments, the development support service 436 assists operators of the hub and third parties in building and/or adapting new products or services for access via the hub.

In some embodiments, the different components (e.g., hardware and/or software modules) of the facility interact in many ways to create a unique healthcare service that use or depend on the many interactions and interdependencies of the components. In some embodiments, the components provide a sequence of care, in which each step depends on the interactions of many of the other components.

For example, initially a user submits a query (e.g., a question) via the library component 414. To enable a doctor to provide an answer to the question that is personalized to the user, profile and patient chart component 413 provides to the doctor information (e.g., up-to-date medical records and/or demographics) specific to the user. In some embodiments, the profile and patient chart component 413 also enables the facility to ask questions that are relevant to the user's query and that have not been answered already. In so doing, the library component 414 also supports updating and/or supplementing the health profile of the user, as a step before sending new question(s) of the user to doctor(s) for a personalized answer.

In accordance with the above example, the directory component 418 supports the library component 414 by providing doctor-specific information about the expertise needed to answer a question, so that the question can be properly routed and placed in question queue(s) of the doctor(s) who have the expertise matched for that question, based either on their specialty certification, or on their set of known-for topics that are recorded on the doctors' profiles. In the other direction, the library component 414 supports the directory component 418 by recording on the provider profiles the specific topic(s) that a doctor is “known for,” based on positive peer review(s) that doctors receive for their answers. Illustratively, each positive peer review of an answer to a user-submitted question gives a corresponding doctor a “known-for” credit for the topics of the question.

In the same example, the library component 414 also supports the symptom triage component 415, when doctors provide answers to users' questions that direct individual users to the need for care for any particular topic or situation described in their question(s). The symptom triage component 415 interacts with the directory component 418 to identify available provider(s) and thereby directs a triaged user to a particular level of care. The directory component 418 depends not only on the library component 414 for information about each provider (e.g., from their peer review of answers, from the user feedbacks on their answers, and/or the like), but also on the profile and patient chart component 413, which provides information including the feedback from users who receive care from the doctors.

With continued reference to the example above, the profile and patient chart component 413 maintains patient health profiles that include the questions individual patient users asked. The profiles not only record the answers for the users, but also give the doctors access to the health concerns of patient users during telemedicine consultation. In some embodiments, telemedicine and/or virtual care can only occur with the use of a common patient chart and health profile, where the doctor can see prior consultations by other, possibly unrelated doctors, as well as the users' questions and answers that reveal their health concerns and the answers from other doctors the patient user has received in past.

In accordance with this example, the scheduling component 417, telemedicine and messaging component 416, prescription/laboratory order component 420, and patient engagement component 421 also interact with other components accordingly. All the inter-component interactions and dependency enables the integration of each functional step with uniform reporting and access in a universally accessible location for all doctors or other professionals who address healthcare needs of the users.

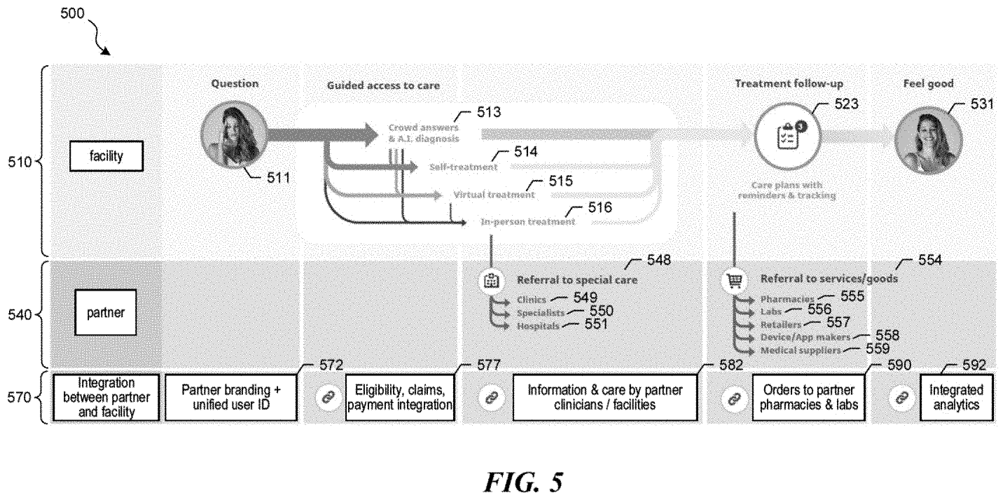

FIG. 5 is an integration diagram showing details of how the facility enables the hub to be integrated with the efforts of partners and/or other third parties. The shown integration 500 includes interactions 510 performed automatically by the facility and/or by operators of the facility; interactions 540 performed by or on behalf of a partner; and integration items 570 used to tie these interactions together. First, a patient user of the facility poses a question 511. The facility uses integration aspect 572 to add branding for the partner to the user interface in which the question is posed, and access information from the partner about the user's identity. In some embodiments, informational answer(s) to the question 511 can be crowdsourced from credentialed medical experts and made discoverable by search to the user or other users.

In response to the question, the facility provides guided access to care, including answers to the question previously supplied by providers and/or contemporaneously provided by providers, and/or an automatic diagnosis 513. The facility also provides self-treatment options 514 springing from those answers/automatic diagnosis. The facility also provides virtual treatment 515. In some embodiments, the facility uses machine-learning or other Al based algorithms to output probable diagnoses and recommend clinically appropriate, yet cost-effective option(s) for the user to potentially act on as next step. The facility also provides options for in-person treatment 516. In some embodiments, in providing these responses to the question, the facility relies on eligibility, claims, and payment integration elements 577. In some embodiments, the facility uses predefined ontology and/or directory to assist the user to identify one or more providers based on a question or topic.

In many cases, the in-person treatment options provided by the facility involve a referral 548 to specialty care by the partner or providers affiliated with the partner, such as clinics 549, specialists 550, and hospitals 551. This is assisted by integration elements 582 containing information about clinicians and facilities provided by or affiliated with the partner. In some embodiments, the facility enables the user (e.g., by providing a graphical user interface) to schedule an appointment to see one or more providers in person, and/or interact with the provider(s) via video and/or text chat using the user's end device. In some embodiments, the facility enables the user to receive physicians orders, which can include prescriptions sent electronically to local and mail order pharmacies, orders sent to local lab tests or imaging centers, referrals to see a different provider, and/or tasks for the user patient to complete at some frequency and schedule after a physician visit.

The facility also provides treatment follow-up 523, such as care plans with reminders and tracking. In some embodiments, the facility sends timely reminders electronically to the patient user to encourage completion of the prescribed or planned tasks. The facility can track whether the patient user completed the tasks or not (e.g., based on self-reporting), which in turn can be viewed by the patient's physician(s) in the future to assess the patient's adherence.

In some embodiments, the facility provides the user with options to purchase and/or consume related healthcare goods or services (e.g., generic OTC drug, at home test, OTC treatment or device, etc.) These can involve referrals 554 to services or goods provided by the partner, such as pharmacies 555, labs 556, retailers 557, device or application makers 558, and medical suppliers 559. In some embodiments, this is assisted by integration element 590, which provides details about how to issue orders to pharmacies and labs operated by or affiliated with the partner.

The facility also provides features during a subsequent period 531 during which the patient is free of medical complaints. These are assisted by integrated analytics integration elements 592.

In some embodiments, the facility can support integration with an unlimited number of different partners, including sets of partners that are exposed to different users or different groups of users.

In some embodiments, the facility enables the user to personalize some or all aspects of the above mentioned interactions to inform a more responsive, more accurate, and/or more cost-effective user experience based on a unified, up-to-date medical record of the user.

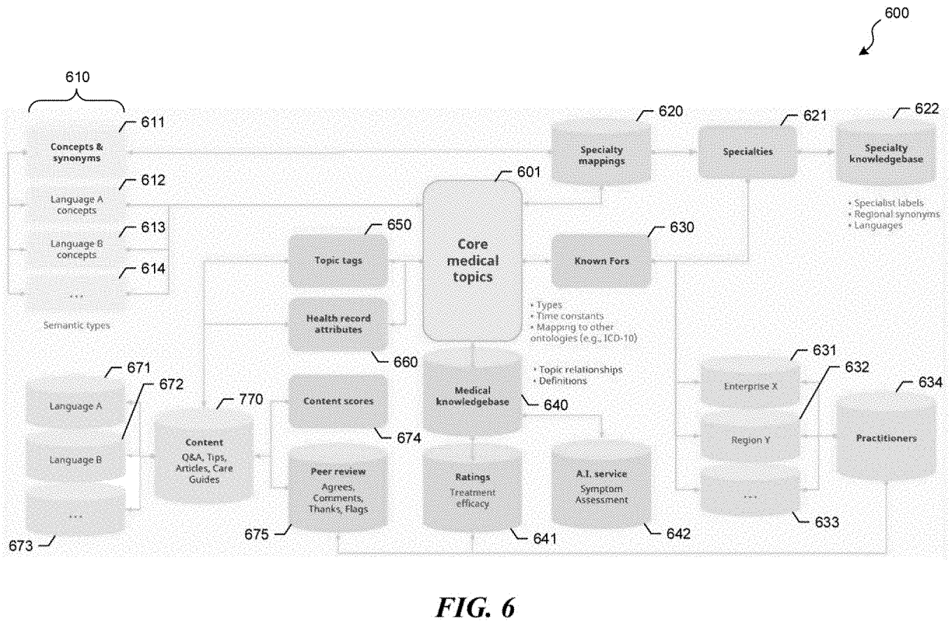

FIG. 6 is an ontology diagram showing a consumer health natural language ontology used by the facility in some embodiments. The ontology 600 is focused on core medical topics 601. These are expressed using semantic types 610, such as concepts and synonyms 611, language A concepts 612, language B concepts 613, and other semantic types 614. The core medical topics also use specialty mappings 620 which connect medical concepts to different specialties 621, represented in a specialty knowledgebase 622 that in some embodiments includes specialist labels, regional synonyms, and languages. The core medical topics also relate to “known fors,” such as types, time constraints, and mappings to other ontologies like ICD-10. Also involved are practitioners 634, such as those associated with a particular enterprise 631, region 632, or other basis for grouping 633. The core medical topics are also the subject of a medical knowledgebase 640 which is linked to treatment efficacy ratings 641 and a symptom assessment artificial intelligence service 642. The core medical topics also relate to topic tags 650 and health record attributes 660. These are linked to content 670, such as questions and answers, tips, articles, and care guides. These have content scores 674, as well as peer reviews 675, which can include concurrences, comments, thanks, and flags. The content can be expressed in any of a number of natural languages 671-673.

It will be appreciated by those skilled in the art that the above-described facility may be straightforwardly adapted or extended in various ways. While the foregoing description makes reference to particular embodiments, the scope of the invention is defined solely by the claims that follow and the elements recited therein.

We claim:

1. A computer-implemented method for accessing multiple healthcare services via a single computer-executable application, comprising:
in response to receiving a health-related request from a user:
providing one or more informational answers to the request;
presenting one or more treatment options based, at least in part, on user symptoms indicated in the request; and
connecting the user with at least one healthcare service provider;
wherein the providing, presenting, and connecting are based, at least in part, on up-to-date medical record of the user.

2. The method of claim 1, wherein the one or more informational answers are crowdsourced from credentialed professionals and searchable to the user or another user.

3. The method of claim 1, wherein the one or more treatment options include at least one of a self-treatment option, virtual treatment option, or in-person treatment option.

4. The method of claim 1, further comprising providing the user access to one or more orders made by the at least one healthcare service provider.

5. The method of claim 1, further comprising sending one or more reminders electronically to the user to encourage completion of tasks prescribed or planned by the at least one healthcare service provider.

6. The method of claim 1, further comprising presenting a user interface for the user to acquire healthcare goods or services related to the request.

7. One or more instances of computer-readable media having collective contents configured to cause a computing system to perform a method, the method comprising:
receiving a symptom triage request from a first user;
providing to the first user a symptom triage response that is responsive to the received symptom request,
receiving a prescription fulfillment request from a second user; and
notifying the second user of prescription fulfillment that is responsive to the received prescription fulfillment request.

8. The one or more instances of computer-readable media of claim 7, the method further comprising, before providing the symptom triage response to the first user, skinning the symptom triage response to identify it as coming from a separate partner organization.

9. The one or more instances of computer-readable media of claim 7, the method further comprising consulting a patient medical chart for the first user to obtain healthcare information for the first user,
and wherein the provided symptom triage response is based at least in part on the obtained healthcare information for the first user.

10. The one or more instances of computer-readable media of claim 7, the method further comprising:
receiving a healthcare scheduling request from a third user; and
providing to the third user a healthcare scheduling response that is responsive to the received healthcare scheduling request.

11. The one or more instances of computer-readable media of claim 7, the method further comprising:
receiving a healthcare directory request from a fourth user; and
providing to the fourth user a healthcare directory response that is responsive to the received healthcare directory request.

12. The one or more instances of computer-readable media of claim 7, the method further comprising:
receiving a healthcare billing request from a fifth user; and
providing to the fifth user a healthcare billing response that is responsive to the received healthcare billing request.

13. The one or more instances of computer-readable media of claim 7, the method further comprising:
receiving a healthcare claims request from a sixth user; and
providing to the sixth user a healthcare claims response that is responsive to the received healthcare claims request.

14. The one or more instances of computer-readable media of claim 7, the method further comprising:
receiving a healthcare library request from a seventh user; and
providing to the seventh user a healthcare library response that is responsive to the received healthcare library request.

15. The one or more instances of computer-readable media of claim 7, the method further comprising:
receiving a telemedicine request from a eighth user; and
providing to the eighth user a telemedicine response that is responsive to the received telemedicine request.

16. The one or more instances of computer-readable media of claim 7, the method further comprising:
receiving a healthcare practice administration request from a ninth user; and
providing to the ninth user a healthcare practice administration response that is responsive to the received practice administration library request.

17. The one or more instances of computer-readable media of claim 7, the method further comprising:
receiving a healthcare analytics request from a tenth user; and
providing to the tenth user a healthcare analytics response that is responsive to the received analytics library request.

18. The one or more instances of computer-readable media of claim 7, the method further comprising:
receiving a training request from an eleventh user; and
providing to the eleventh user a training response that is responsive to the received training request.

19. The one or more instances of computer-readable media of claim 7, the method further comprising:
receiving a support request from a twelfth user; and
providing to the twelfth user a support response that is responsive to the received support request.

20. The one or more instances of computer-readable media of claim 7, the method further comprising:
receiving a healthcare module development support request from a thirteenth user; and
providing to the thirteenth user a healthcare module development support response that is responsive to the received healthcare module development support request.

21. The one or more instances of computer-readable media of claim 7, the method further comprising:
receiving a healthcare referral request from a fourteenth user; and
providing to the fourteenth user a healthcare referral response that is responsive to the received healthcare referral request.

22. The one or more instances of computer-readable media of claim 21, the method further comprising:
identifying a partner with whom the fourteenth user is associated; and
determining the healthcare referral response based at least in part with the determined partner.

23. The one or more instances of computer-readable media of claim 21 wherein the healthcare referral response is a referral to healthcare services.

24. The one or more instances of computer-readable media of claim 21 wherein the healthcare referral response is a referral to a particular healthcare provider.

25. The one or more instances of computer-readable media of claim 21 wherein the healthcare referral response is a referral to healthcare goods.

26. A method in a computing system, comprising:
receiving a symptom triage request from a first user;
providing to the first user a symptom triage response that is responsive to the received symptom request,
receiving a prescription fulfillment request from a second user; and
notifying the second user of prescription fulfillment that is responsive to the received prescription fulfillment request.

27. The method of claim 26, further comprising, before providing the symptom triage response to the first user, skinning the symptom triage response to identify it as coming from a separate partner organization.

28. The method of claim 26, further comprising consulting a patient medical chart for the first user to obtain healthcare information for the first user, and wherein the provided symptom triage response is based at least in part on the obtained healthcare information for the first user.

29. The method of claim 26, further comprising:
receiving a healthcare scheduling request from a third user; and
providing to the third user a healthcare scheduling response that is responsive to the received healthcare scheduling request.

30. The method of claim 26, further comprising:
receiving a healthcare directory request from a fourth user; and
providing to the fourth user a healthcare directory response that is responsive to the received healthcare directory request.

31. The method of claim 26, further comprising:
receiving a healthcare billing request from a fifth user; and
providing to the fifth user a healthcare billing response that is responsive to the received healthcare billing request.

32. The method of claim 26, further comprising:
receiving a healthcare claims request from a sixth user; and
providing to the sixth user a healthcare claims response that is responsive to the received healthcare claims request.

33. The method of claim 26, further comprising:
receiving a healthcare library request from a seventh user; and
providing to the seventh user a healthcare library response that is responsive to the received healthcare library request.

34. The method of claim 26, further comprising:
receiving a telemedicine request from a eighth user; and
providing to the eighth user a telemedicine response that is responsive to the received telemedicine request.

35. The method of claim 26, further comprising:
receiving a healthcare practice administration request from a ninth user; and
providing to the ninth user a healthcare practice administration response that is responsive to the received practice administration library request.

36. The method of claim 26 further comprising:
receiving a healthcare analytics request from a tenth user; and
providing to the tenth user a healthcare analytics response that is responsive to the received analytics library request.

37. The method of claim 26, further comprising:
receiving a training request from an eleventh user; and
providing to the eleventh user a training response that is responsive to the received training request.

38. The method of claim 26, further comprising:
receiving a support request from a twelfth user; and
providing to the twelfth user a support response that is responsive to the received support request.

39. The method of claim 26, further comprising:
receiving a healthcare module development support request from a thirteenth user; and
providing to the thirteenth user a healthcare module development support response that is responsive to the received healthcare module development support request.

40. The method of claim 26, further comprising:
receiving a healthcare referral request from a fourteenth user; and
providing to the fourteenth user a healthcare referral response that is responsive to the received healthcare referral request.

41. The method of claim 40, further comprising:
identifying a partner with whom the fourteenth user is associated; and
determining the healthcare referral response based at least in part with the determined partner.

42. The method of claim 40, wherein the healthcare referral response is a referral to healthcare services.

43. The method of claim 40, wherein the healthcare referral response is a referral to a particular healthcare provider.

44. The method of claim 40, wherein the healthcare referral response is a referral to healthcare goods.
